(19) United States
(12) Patent Application Publication
(10) Pub. No.: US 2021/0189455 A1
(43) Pub. Date: Jun. 24, 2021

(54) METHODS FOR SAMPLE PREPARATION AND MICROBIOME CHARACTERISATION

(71) Applicant: MICROBA IP PTY LIMITED, Brisbane, Queensland (AU)

(72) Inventors: Gene William TYSON, Brisbane (AU); Philip HUGENHOLTZ, Brisbane (AU); Nicola Zoe ANGEL, Woolloongabba (AU)

(73) Assignee: MICROBA IP PTY LIMITED, Brisbane, Queensland (AU)

(21) Appl. No.: 17/252,375

(22) PCT Filed: Jun. 15, 2019
(86) PCT No.: PCT/AU2019/050618

§ 371 Date: Dec. 15, 2020

(30) Foreign Application Priority Data
Jun. 15, 2018 (AU) 2018902147

(51) Int. Cl. C12Q 1/24; B01L 3/00; C12Q 1/10; C12Q 1/6806; A61B 10/00

(52) U.S. Cl. CPC C12Q 1/24; B01L 3/5029; C12Q 1/10; B01L 2300/105; A61B 10/0038; B01L 2300/0845; C12Q 1/6806

(57) ABSTRACT

Disclosed herein are methods and kits for performing microbiome analysis in the field of microbiology. More specifically, disclosed herein are methods and kits for remote sample collection and sample preservation so that analysis may be performed on the sample in a laboratory.

### FIELD OF THE INVENTION

This invention relates generally to the field of microbiology. More particularly, the invention relates to methods and kits for performing microbiome analysis in the field of microbiology. In addition, the invention also relates to methods and kits for remote sample collection and sample preservation so that analysis may be performed on the sample in a laboratory.

### BACKGROUND OF THE INVENTION

Any reference in this specification to any known matter or prior publication (or information derived from it), is not an acknowledgement, admission or suggestion that the known matter, prior publication, or any information therefrom forms part of the common general knowledge in the field of endeavor to which this specification relates.

The microbiome is an ecological community of commensal, symbiotic and pathogenic microorganisms, including bacteria, archaea, fungi, viruses, and protists. The human body is reported to comprise over 10% more microbial cells than human cells (see, Sender et al, 2016). However, techniques and methods for the characterization of the human microbiome are still in early stages due to limitations in sample processing techniques, genetic analysis techniques, and resources for processing large amounts of data. Traditional characterization techniques are generally limited to classical phenotypic techniques (see, Clarridge, 2004; and Huse, 2010).

With the improvement of high-throughput sequencing technology, the ability to profile complex microbial communities without the need to individually culture organisms has increased dramatically. Sequencing studies utilizing the highly conserved 16S ribosomal RNA (rRNA) gene have substantially changed our understanding of phylogeny and microbial diversity. This technology has become a staple for profiling microbial communities and their abundancies from soil to humans, including the human microbiome. However, the 16S rRNA methods are not without their limitations; the community profiles are biased by primer choice, and the taxonomic annotation is based on sequence similarity of existing 16S rRNA gene fragments with a representative sequence from an experiment-specific cluster of sequences (termed an operational taxonomic unit (OTU)). While 16S rRNA sequences are good biomarkers because of their ubiquity, OTUs are typically analyzed at the family or genus level due to their high conservation, and can be identical across species or strains. In addition, functional genes from the community are not directly sequenced, but rather imputed based on known knowledge from close type strain relatives. Thus, due to horizontal gene transfer and the existence of numerous bacterial strains with substantial gene content differences, the lack of any direct gene identification potentially limits our understanding of the microbiome (see, Poretsky, 2014; Konstantinidis, 2007; and Konstantinidis, 2013).

Several products are available to allow a customer to collect a microbiome sample and send to a laboratory in order to obtain information that relates to the composition of their gut microbiome. However, during the time between the customer collecting the sample and the sample being received by the laboratory typically spans at least a number of days. During this time, the important nucleic acid material starts to degrade making the results unsuitable for processing, or at best, unreliable. Typically, liquid processing and pre-processing reagents are included in the sample collection containers, such that customer mixes the sample with the reagent in order to initate reactions (cell lysis and nucleic acid stabilization) to preserve the integrity of the nucleic acid material within the sample.

Although the inclusion of a sample processing reagent (e.g., lysis buffer) is generally in the art viewed as having an important function, consumer complience when collecting into such a processing reagent is comparatively low. That is, many returned samples fail quality control (QC) during the nucleic acid sequencing process.

Futhermore, other problems observed by the present inventors include that the small tube sizes are generally found difficult to handle with many outside of the lab sample collection kits being notoriously split. Accordingly, the commercially available kits for remote sample collection generally include two containers to safeguard against such spillages, which further increases the overall cost per sample to be analysed.

In addition, using chemical products to lyse cells in the samples presents as an additional chemical safety hazard for customers. In addition, these methods provides a higher risk for the transport of the sample in instances relating to tube failures or leaks.

There are also problems from a technical perspective, as a well-defined reagent to sample ratio is required. There is no way to address insufficient nucleic acid material being provided in the sample once the processing reaction has already commenced and the sample is being provided in liquid form, as the sample amount cannot be altered in the lysate after it is received at the laboratory.

Furthermore, the better performing sample processing reagents and DNA stabilisation chemicals are very expensive, which adds considerable cost to the sample kit.

### SUMMARY OF THE INVENTION

The present invention was predicated, at least in part, on the realization by the present inventors that drying a microbiome sample before or during transport to a processing facility, allows for improved sample processing prior to nucleic acid sequencing.

In this regard, in one aspect the invention provides a use of a sample collection device in a nucleic acid sequencing process, wherein the sample collection device comprises: (i) a container; (ii) a sample collection element comprising a support body and a collection portion; and (iii) a sample drying agent. Preferably, the support body comprises a longitudinal extension. The length of the longitudinal extension is typically selected from: between about 2 cm and about 20 cm; between about 3 cm and about 18 cm; and between about 6 cm and about 16 cm. The longitudinal extension generally has a thickness or diameter in a section that is perpendicular to the central axis thereof, comprising between about 0.5 mm and about 5 mm; between about 1 mm and about 3 mm; or between about 1.5 mm and about 2.5 mm.

Preferably, the sample drying agent is located at least partially within the container.

In some preferred embodiments, the container does not comprise any processing reagents (e.g., lysis buffers, PCR buffers, preservatives, etc.).

Typically, the nucleic acid sequencing process comprises a whole genome sequencing method.

In some embodiments, the collection portion comprises a plurality of elongated fibres. The elongated fibres are substantially composed of a suitable synthetic or artificial material, or a combination thereof.

In some embodiments, the synthetic material is selected from at least one of: nylon, rayon, polyester, polyamide, carbon fibre, alginate, and a mixture thereof. In some preferred embodiments, the synthetic material is substantially composed from nylon.

In some alternative embodiments, wherein the elongated fibres are substantially composed of a natural material. Non-limiting examples of a suitable natural material include cotton, silk, and/or a mixture thereof.

In some embodiments, the elongated fibres have hydrophilic properties.

In some embodiments, the plurality of fibres are arranged as a layer having a substantially uniform thickness.

In some embodiments, the fibres are deposited on the collection portion of the device by flocking in an ordered arrangement of the fibres normal to a non-absorbent surface of the collection portion.

In some embodiments, the sample collection device is configured for collecting a fecal sample from the subject (e.g., collecting stool from used toilet paper). In some alternative embodiments, sample collected from the subject may be selected from a fecal sample, saliva sample, blood sample, skin sample, plasma/serum sample, oral sample, genital sample, nasal sample, eye sample, and ear sample.

In some embodiments, the sample collected using the device is used to characterise the gut microbiome.

In another aspect, the invention provides a use of a sample collection device comprising a sample drying agent in the manufacture of a kit for analyzing or otherwise interrogating nucleic acid material in a microbiome sample of a subject.

Typically, the nucleic acid material is derived from a microorganism present in a microbiome of a subject.

In another aspect, the invention provides a method of preparing a sample for nucleic acid sequencing, the method comprising:

providing a sampling kit to a subject at a remote location, wherein the sampling kit comprises a sample collection device comprising: (i) a container; (ii) a collection element comprising a support body and a collection portion; and (iii) a sample drying agent;

receiving the container with a sample from the subject; and

sequencing at least a portion of a nucleic acid in the sample.

In some embodiments, the container is free of any sample processing reagents and/or chemicals (e.g., lysis buffers, PCR buffers, preservatives, etc.), and configured to receive a sample from a collection site of the subject.

In some embodiments, the nucleic acid sequencing comprises a whole genome sequencing method.

Typically, the nucleic acid is derived from at least one microorganism within the sample.

In some preferred embodiments, the sample is a fecal sample, and the microorganism is present in the gut microbiome of the subject.

Typically, the support body of the device comprises a longitudinal extension.

Preferably, the sample drying agent is located at least partially within the container. In some embodiments, the sample drying agent functions to dry or dehumidify the sample present in the container. The sample drying agents of the invention are typically substantially composed of a hygroscopic substance. Although in some embodiments the sample drying agent is in solid form, other forms are also envisaged (and may work through other principles, such as chemical bonding of water molecules). By way of an illustrative example, the sample drying agent may be substantially composed of a composition selected from: activated alumina, aerogel, benzophenone, bentonite clay, calcium chloride, calcium oxide, calcium sulfate, cobalt(II) chloride, copper(II) sulfate, lithium chloride, lithium bromide, magnesium sulfate, magnesium perchlorate, potassium carbonate, potassium hydroxide, silica, sodium, sodium chlorate, sodium chloride, sodium hydroxide, sodium sulfate, sucrose, and sulfuric acid. In some preferred embodiments, the sample drying agent is substantially composed of silica. In some embodiments the sample drying agent is provided in a sachet, bag or mesh. Typically, the sample drying agent is fully or at least partially housed inside the container or in another useful position. For example, in some embodiments the sample drying agent (e.g., sachet of silica gel) is housed in the lid of the container, and is in fluid communication with the sample collection portion of the device.

In some embodiments, the collection portion comprises a plurality of elongated fibres. Suitably, the fibres have hydrophilic properties. In some embodiments, the plurality of fibres are arranged as a layer having a substantially uniform thickness. The elongated fibres are typically deposited on the collection portion by flocking on an ordered arrangement of the fibres normal to the non-absorbent surface.

In some preferred embodiments, the sample collection device is configured for collecting a fecal sample from the subject. Typically, the sample collected using the device is used to characterise a microbiome.

In yet another aspect, the present invention comprises a method of preparing a sample for nucleic acid sequencing, the method comprising:

providing a sampling kit to a subject at a remote location, wherein the sampling kit comprises a sample collection device comprising (i) a container; (ii) a sample collection element comprising a support body and a collection portion; and (iii) a sample drying agent; wherein the sample drying agent is sufficient to dry the sample; and

receiving the container comprising the dried sample at a sample sequencing facility.

In yet still another aspect, the invention comprises a method of preparing a sample for nucleic acid sequencing, the method comprising:

providing a sampling kit to a subject at a remote location, wherein the sampling kit comprises a sample collection device comprising: (i) a container; (ii) a sample collection element comprising a support body and a collection portion; and (iii) a sample drying agent;

receiving the sample container with a sample from the subject; and

resuspending the sample in buffer.

In yet another aspect, the present invention comprises a method of characterising the composition of a microbiome in a subject, the method comprising:

providing a sampling kit to a subject at a remote location, wherein the sampling kit comprises a sample collection device comprising: (i) a container; (ii) a collection element comprising a support body and a collection portion; and (iii) a sample drying agent;

receiving the sample container containing a sample from the subject;

sequencing nucleic acid content from the microorganism portion of the sample, to generate a microbiome sequence dataset;

identifying a set of microorganisms present in the microorganism portion of the sample, based on the microbiome sequence dataset;

generating an analysis based on the set of microorganisms present in the microorganism portion of the sample; and

communicating the analysis to the subject.

Preferably, the container is free from any sample processing reagents and/or chemicals (e.g., lysis buffers, PCR buffers, preservatives, etc.).

In some embodiments, the microbiome is a gut microbiome.

In some preferred embodiments the nucleic acid sequencing comprises whole genome nucleic acid sequencing. In some of the same embodiments and other embodiments, the nucleic acid material is derived from a microorganism.

In some preferred embodiments, the sample is a fecal sample, and the microorganism is present in the gut microbiome of the subject.

In yet still another aspect, the present invention provides a microbiome characterisation kit, the kit comprising:

a sample collection device that comprises: (i) a container that is free from any sample processing reagents and/or chemicals (e.g., lysis buffers, PCR buffers, preservatives, etc.); (ii) a sample collection element comprising a support body and a collection portion; and (iii) a sample drying agent.

In some embodiments, the kit further comprises instructions for collecting a sample that comprises a microorganism derived from the gut microbiome of the subject. In some embodiments of this type, the kit also contains a Bristol Stool Chart.

Preferably, the kit further comprises a return envelope or parcel for return of the sample to a nucleic acid sequencing facility.

A method for analysing a microbiome of a subject, comprising:

providing a sampling kit to a subject at a remote location, wherein the sampling kit comprises: (i) a container that is free from any sample processing reagents and/or chemicals (e.g., lysis buffers, PCR buffers, preservatives, etc.); (ii) a sample collection element comprising a support body and a collection portion; and (iii) a sample drying agent;

receiving the sample container with the sample from the collection site of the subject;

generating a microbiome sequence dataset based upon sequencing nucleic acid content of at least one microorganism present in the sample;

identifying a set of microorganisms represented in the microorganism portion based upon performance of a mapping operation on portions of microbiome sequence dataset;

generating an analysis based upon a set of features related to the microorganism portion; and

transmitting information derived from the analysis to the subject.

### BRIEF DESCRIPTION OF THE FIGURES

An example of the present invention will now be described with reference to the accompanying drawings, in which:

FIG. 1 is a flowchart illustrating a process described herein.

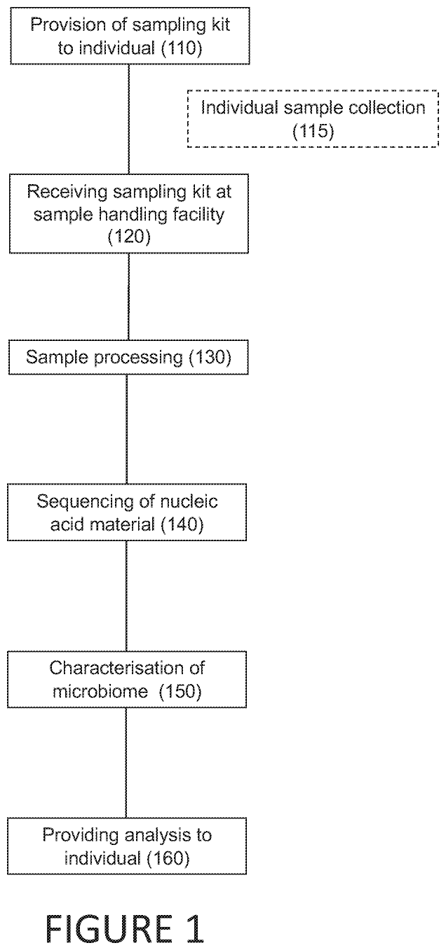

FIG. 2 is a graphical representation of a principal component analysis plot of Hellinger transformed species profiles. Samples from each subject are assigned a unique colour and each stabilization technique a unique shape. Green: subject 1; yellow: subject 2; purple: subject 3; orange: subject 4; blue: subject 5.

FIG. 3 provides a graphical representation of the species profiles of the five subjects. Different colours correspond to the mean abundance of species determined across all six replicate samples. The light and dark blue bars at the bottom of each bar plot indicate the mean percentage of unmapped and unassigned reads, respectively. Species are sorted from most to least abundant based on the baseline “freeze” samples. The light grey bar at the top of each bar plot indicates species where the maximum mean abundance <0.5% across all stabilization techniques. The same colour between subjects does not necessarily correspond to the same species. Only subjects 4 and 5 were analysed under the DNA/RNA-Shield condition.

FIG. 4 provides a graphical representation of species profiles for the five subjects explicitly shown for each replicate sample. Colours are the same as in FIG. 2.

FIG. 5 provides bubble plot representations illustrating the species profiles for the five participants. Shown is the (A) relative abundance and (B) relative abundance minus the mean species abundance in the frozen sample controls. Circle sizes represent relative abundance of taxa. Subjects are depicted as P1 to P5. (B) All samples with abundance>mean abundance in frozen samples are shown as circles. Negative values (samples with abundance<mean abundance in frozen samples) are not shown.

FIG. 6 provides a graphical representation of the diversity of the replicate species profiles for the different stabilisation techniques and control frozen samples aggregated over all five subjects. The box-and-whisker plot shows the lower and upper quartiles as a box, the median value as a line within the box, 1.5 times the interquartile range as whiskers, and outliers as crosses.

FIG. 7 provides graphical representations of the diversity of the replicate species profiles, including (A) Bray-Curtis diversity; (B) Hamming distance; and (C) Sorensen diversity; for the different stabilization techniques and the samples frozen at time zero for each of the five participants.

FIG. 8 provides a graphical representation of the diversity of species profiles for the different stabilization techniques, as compared to profiles from the control frozen samples taken at timepoint zero. Results are aggregated over all five subjects.

FIG. 9 provides graphical representations of the diversity of species profiles for the different stabilization techniques compared to profiles from the frozen samples taken at time zero. Results are shown for each of the five subjects.

### DETAILED DESCRIPTION OF THE INVENTION

The following description of the embodiments of the invention is not intended to limit the invention to these embodiments, but rather to enable any person skilled in the art to make and use this invention.

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “a sample” means one sample or more than one sample. Thus, for example, the term “fecal sample” also includes a plurality of fecal samples.

As used herein, “and/or” refers to and encompasses any and all possible combinations of one or more of the associated listed items, as well as the lack of combinations when interpreted in the alternative (or).

Further, the term “about”, as used herein when referring to a measurable value such as an amount, dose, time, temperature, activity, level, number, frequency, percentage, dimension, size, amount, weight, position, length and the like, is meant to encompass variations of ±20%, ±10%, ±5%, ±1%, ±0.5%, or even ±0.1% of the specified amount, dose, time, temperature, activity, level, number, frequency, percentage, dimension, size, amount, weight, position, length and the like.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “drying” is synonomous with “dehumidifying” and “desiccating” and refers to the removal of moisture from an environment, typically for preservation.

As used herein, the term “microbiome” refers to the gut microbiome. The gut microbiome (or human gut microbiome) may be understood as the aggregate of microorganisms that reside on the surface in the gastrointestinal tracts of humans. The human microbiome is comprised of bacteria, fungi, viruses, and archaea. At least some of these organisms perform tasks that are useful for the human host. Under normal (i.e., healthy) circumstances, these microorganisms do not cause disease to the human host, but instead participate in maintaining health. Hence, this population of organisms is frequently referred to as “normal flora.”

As used herein, the term “sample” is to mean any source suspected to contain a nucleic acid component to be characterised or identified. A sample can be “neat” or can be diluted with an appropriate buffer or solvent. Currently preferred samples include, but are not limited to, any biological specimen suspected to comprise a nucleic acid component. Samples suitable for use in the claimed invention include, but not limited to, a fecal sample. As used herein, the term “component” is intended to mean any identifiable or detectable substance, or a substance susceptible to separation from other substances in a sample. Preferred components include, but are not limited to, chemical and biochemical moieties, such as nucleic acids, proteins, and peptides.

The terms “subject,” “host,” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylumn Chordata including primates (e.g., humans, monkeys, and apes, and includes species of monkeys such as from the genus Macaca (e.g., cynomologus monkeys such as Macaca fascicularis, and/or rhesus monkeys (Macaca mulatta) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes), rodents (e.g., mice, rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars, etc.), marine mammals (e.g., dolphins, whales), reptiles (e.g., snakes, frogs, lizards, etc.), and fish. A preferred subject is a human.

Persons skilled in the art will appreciate that numerous variations and modifications will become apparent. All such variations and modifications which become apparent to persons skilled in the art, should be considered to fall within the spirit and scope of the invention broadly before described.

Thus, for example, it will be appreciated that features from different examples above may be used interchangeably where appropriate.

### 2. Sample Collection Device

The nucleic acid sequencing processes and methods described herein typically require the use of a sample collection device. The sample collection devices typically comprise: (i) a container; (ii) a sample collection element comprising a support body and a collection portion; and (iii) a sample drying agent.

The sample collection device is preferably configured to facilitate reception of samples from a subject in an invasive and/or non-invasive manner.

In some embodiments, the container comprises a vial, tube, or bag that is configured to receive a sample from a region of a subject's body, and/or any other suitable sample reception element. In some embodiments, the container comprises a substantially cylindrical test-tube.

Suitably, the upper open end of the container has a collar for receiving a closure means. In some embodiments, the closure means comprises a closing cap or stopper that is removably mountable at the access opening, for selectively closing the container. Typically, the cap or stopper is shaped so that it can engage, for example, by snap-engaging, with the collar of the container. In some preferred embodiments, the closing cap or stopper is attached to the support body of the sample collection element, at the opposite end to the collecting portion.

The container, closing cap, and/or the support body can each be made of a plastic material. Suitable materials include (but are not limited to) polystyrol, polystyrene, or polypropylene and/or any other material suitable for use with the specific sample to be collected or generally suited to use with biological materials or materials of biological origin. In some preferred embodiments, the container, closing cap, and/or the support body can be sterilized.

The collection device may further comprise a sealed packaging in which the container, closing cap or stopper, and the sample collection element, can be housed before use in collecting a sample.

Preferably, the support body comprises a longitudinal extension. The length of the longitudinal extension is typically selected from between about 2 cm and about 20 cm; between about 3 cm and about 18 cm; and between about 6 cm and about 16 cm. The longitudinal extension generally exhibits a thickness or diameter in a section that is perpendicular to the central axis thereof, comprised between about 0.5 mm and about 5 mm; between about 1 mm and about 3 mm; or between about 1.5 mm and about 2.5 mm. In some embodiments, longitudinal extension is substantially composed of a synthetic material (e.g., plastic).

Typically, the collection portion is located at one end of support body. In some preferred embodiments, the collection portion exhibits any shape suitable for the type of sample to be collected. In some embodiments, the support body can be provided with an intermediate weakened portion to facilitate a selective breaking of the body itself in an intermediate position between the two ends of the longitudinal extension. This configuration allows for the insertion of the collection portion into a container for transport, or processing after transport.

The collection portion is generally conformed as a swab. In some embodiments, the collection portion includes an absorbent material portion that comprises for example, a layer of fiber, for collecting a biological sample (e.g., a microbiome sample) to be analysed. In some embodiments of this type, the collecting portion is flocked, by way of flocking a plurality of fibres on the sample collecting end of the body. The fibres flocked on the sample collecting end can be made of hydrophilic or non-hydrophilic material, but the collecting portion is hydrophilic by capillary effect of the overall fibre structure. The collecting portion typically comprises a substantially continuous and substantially homogenous layer of a plurality of fibres having an ordered arrangement, each made of a substantially absorbent material (suitable for collecting a fluid, semi-fluid, or solid sample) or a non-absorbent material. The fibres are substantially perpendicular at every point of the support body, and substantially parallel to the adjacent fibres. As the fibres are arranged in an ordered plurality of capillary interstices in which a predetermined quantity of the sample, for example, a liquid sample, can be retained, for example, by imbibition. Typically, the tip portion is shaped in a rounded geometry, similar to an ogive. Because of the flocking process, the fibres are generally disposed as a substantially continuous layer of uniform thickness. For example, the fibre typically has hydrophilic properties, and is deposited by means of flocking. The fibre that forms the flocked layer is generally deposited in an oriented manner and anchored to the surface of the tip, being retained by an adhesive. Any adhesive used is preferably water-based: once it dries it enables the fiber to be anchored in a stable manner to the swab and resistant abrasion.

The flocked collecting portion can be configured and dimensioned such as to collect a quantity of sample comprised for example between about 50 μg and about 500 mg, between about 100 μg and about 250 mg, between about 150 μg and 200 mg, or between about 200 μg and about 400 μg. The fibres may be arranged on the support body in a substantially ordered way and in such a way as to form a substantially continuous layer on the collecting portion and/or can be arranged on the collection portion in such a way as to define a plurality of capillary interstices destined to adsorb the liquid sample by capillary action.

In some preferred embodiments, the fibre count (i.e., the weight in grams per 100 linear metres of a single fiber) can be selected from: between about 1 Dtex and 10 Dtex, between about 1.7 Dtex and 3.3 Dtex, and/or the fibres can exhibit a length comprised between 0.6 mm and 3 mm. For example, a fibre of about 0.6 mm length and 1.7 Dtex can be applied by flocking to obtain a fine nap, and a fibre up to 3 mm in length and 3.3 Dtex can be applied to obtain a long nap. The fibres may be arranged by flocking on the collecting potion of the support body with a surface density comprised for example between about 50 fibers per mm² and about 500 fibers per mm²; or between about 100 mm² and about 200 fibres per mm², of surface.

The layer of fibres can define an absorbance capacity for example or at least about 0.5 μL per mm², or at least about 0.6 μL per mm², or at least about 0.7 μL per mm². or at least about 0.75 μL per mm² of surface of the support body.

In some embodiments, the fibres are treated with a surfactant before use for collecting the sample, for example, during manufacture of the sample collection device. The surfactant may be cationic, anionic, non-ionic, or amphoteric. In some preferred embodiments, the surfactant is cationic, for example, benzalkonium chloride (BAC or alkyl-dimethyl-benzylammonium chloride or ADBAC). Alternatively, the cationic surfactant may be a salt having a positive part, constituted by at least a chain of carbon atoms with a quaternary ammonia group, and/or can be a quaternary ammonia salt or can comprise a mixture of ammonia salts. The cationic surfactant can be a mixture of chlorides of alkyl-benzyl-dimethyl. The cationic surfactant can be a mixture of chlorides of alkyl-benzyl-dimethyl ammonium, in which the alkyl group varies from octile (C₈H₁₇—) to octadecyl (C₁₈H₃₇—). In some alternative embodiments, the cationic surfactant could be cetryltrimethyl ammonium bromide (CTAB or hexadecyl trimethyl ammonium bromide). In further alternative embodiments the cationic surfactant may be, but not limited to, benzethonium chloride, cetalkonium chloride, laurtrimonium bromine, myristyltrimethylammonium bromide, cetrimide, cetrimonium bromide, cetylpyridinium chloride or stearalkonium chloride.

In some other embodiments, the collection portion comprises wicking paper (e.g., FTA cards).

Typically, the sample drying agent comprises a chemical composition, organic composition, or inorganic composition that functions to remove moisture from the surrounding (e.g., a closed environment). By way of an illustrative example of an embodiment of this type, the drying element may comprise a sachet, packet or bag containing silica gel. Typically, the sample drying agent is housed at least partially in the container or in another useful position within the closed environment that comprises the sample from the subject. In some embodiments, the sample drying agent comprises any chemical composition that absorbs moisture from its environment. In some preferred embodiments, the sample drying agent is housed within the closing cap or stopper of the container, and is in fluid communication with the interior volume of the container.

In some embodiments, the sampling kit further comprises instructions that are provided to guide a remote subject in providing one or more samples in a dependable manner, guide a remote subject in performing some aspects of sample pre-processing (e.g., with the subject's acknowledgement, in a surreptitious manner without the subject's acknowledgment). For example, instructions for the provision of a sample may include at least one of: instructions specific to one or more of a set of collection sites of the body of a subject; instructions with respect to an amount of sample to be provided by the subject; instructions pertaining to the time(s) of day at which to provide samples; instructions pertaining to behaviors that should be avoided prior to and during sample provision; instructions pertaining to behavior that are encouraged prior to and/during sample provision; instructions regarding correction of an improperly provided sample; instructions regarding storage of a sample prior to transmission to a sequencing facility (e.g., with regard to temperature ranges at which to store a sample, orientation of sample container, etc.); instructions regarding transmission of a sample to a sample sequencing facility; and any other suitable instructions relating to sample provision. The instructions may include instructions to avoid sample contamination. In some embodiments of this type, the instructions may also include additional advice against contact with antiseptics, antibiotic soaps and lotions, and behaviours that could disturb the microbiome of the subject. Instructions may also include instructions regarding packaging of sample containers including collected samples prior to transmission to the sequencing facility (e.g., using a parcel delivery service), and first aid instructions in the event of inappropriate usage.

In some embodiments, the sample collection device further comprises instructions regarding the creation of a user account within an online results platform configured to provide microbiome-derived insights to the subject. Such instructions may include providing a website address by which a subject can set up a user account within an online results platform. Provision of an address can be performed using a messaging client (e.g., a text messaging client, an email messaging client, etc.), using text-based instructions provided within the sampling kit, using a machine-decodable tag (e.g., a QR code, a barcode, an antenna associated with a near field communication NFC device), and/or in any other suitable manner. Instructions may further include instructions regarding account security (e.g., by providing a user name and a password), instructions regarding provision of personal information, instructions regarding associating a user account with an identifying aspect (e.g., registration ID) of a sampling kit, and any other suitable instructions. Information needed from the subject in setting up the user account can be directly input by the subject (e.g., using an input device of an electronic device associated with the subject), and can additionally or alternatively be automatically populated based upon accessing information databases associated with the subject. For instance, information needed in setting up the user account can be populated upon accessing of an electronic health record and/or a social network account (e.g., Facebook account, Linked In account, Twitter account, etc.) associated with the subject, upon receiving permission from the subject.

Any instructions provided may include one or more of: text-based instruction provision; picture-based instruction provision; video-based instruction provision; audio-based instruction provision; and any other suitable form of instruction provision, touch/haptic-based instruction provision.

Preferably, portions of the sampling kit for sample reception (e.g., sample containers) are configured to be delivered back to the sample handling network, the sampling kit can further include a packaging receptacle (e.g., a bubble mailer, an envelope, a parcel, etc.), with or without postage for delivery to the sample handling facility. In some of the same embodiments and other embodiments, portions of the sampling kit can be configured to be picked up by a courier service specifically associated with the sample handling facility (e.g., using a staff of couriers configured to be contacted when a sample from a subject is ready to be picked up), wherein the subject is given instructions to contact the courier service once provision of a sample is complete. The sample delivery process can, however, be facilitated by the sampling kit in any other suitable manner.

Identifying features of the sampling kit can include one or more of: a registration code of characters (e.g., alphanumeric characters), a biological identifier (e.g., a nucleic acid marker with a specific sequence and/or a specific concentration), a machine-readable tag (e.g., QR code, barcode, antenna detectable using a near field communication device, etc.), and/or any other suitable identifier. Variation of elements of the sampling kit can include printed materials and/or digitally stored information (e.g., information stored in memory), and/or can comprise a link, code, or reference to digitally-stored information (e.g., a link to a program, a file, or an application). In some of the same embodiments and other embodiments, the sampling kit may be configured to facilitate instruction provision by way of an electronic device associated with the subject. For instance, a QR code of the sampling kit can be scanned using an electronic device of the subject, wherein the QR code links to an address that includes text and visual instructions for sample provision. In some of the same embodiments and other embodiments, a printed card in the sampling kit can include a website at which instructions for sample provision are provided to the subject. In some embodiments, the instruction card is integral to (i.e., forms part of) the sample container.

### 3. Receiving a Microbiome Sample

As shown in step 110, the sampling kit is typically provided to the individual. Preferably, the sampling kit is provided to a subject located at a location remote from the nucleic acid sequencing facility. Advantageously, this provides a convenient means by which the subject may take a microbiome sample from their own home (or other remote location). The provision of the sampling kits is typically implemented by a sample handling facility, that facilitates the distribution of sampling kits to subjects. The sample handling facility thus functions as a platform from which the sampling kits can be distributed to subjects who are remote from the sequencing facility, and to which sample collection containers including samples from subjects can be returned for processing and analysis. Such embodiments may be advantageous, allowing for subjects to transmit samples directly to the sample handling facility without requiring direct contact between the subjects and a clinical or laboratory-based intermediary staffed with trained personnel for biological sample handling. In some embodiments, the sample handling facility and the sequencing facility are part of the same entity, department, and/or team. For example, the sample handling facility and the sequencing facility may be co-located. In other embodiments, the sample handling facility and the sequencing facility may be separate entities or departments, and/or be located at different geographical locations.

Providing the sampling kit is preferably performed using a parcel delivery service (e.g., postal service, shipping service, mailing service, etc.) accessible to the sequencing facility, such that the sequencing facility can provide the one or more sampling kit(s) to one or more subjects over the parcel delivery service. The sampling kit can additionally or alternatively be provided directly through an entity associated with the sequencing facility, wherein the entity is also trained to facilitate sample reception from a subject. In embodiments of this type, the entity may be selected from a clinical technician, a laboratory technician, a healthcare professional (e.g., doctor, nurse, etc.), a dietician, and any other suitable entity that can facilitate provision of the sampling kit to a subject or facilitate reception of a sample from the subject by way of the sampling kit. However, provision of the sampling kit(s) to the subject(s) can be performed in any other suitable manner.

Preferably, the sampling kit is configured to facilitate reception of biological samples from a subject an invasive or non-invasive manner. In some embodiments, non-invasive manners of sample reception from a subject include the use of the sample collection devices described above and elsewhere herein.

The biological sample obtained from the subject comprises a microbiome portion comprising nucleic acid material from at least one microorganism. In some embodiments, samples from subjects can comprise one or more of fecal samples, saliva samples, blood samples, skin samples, plasma/serum samples (e.g., to enable extraction of cell-free DNA), oral samples, genital samples, nasal samples, eye samples, and ear samples. In some preferred embodiments, the sample is associated with the gut microbiome. In some embodiments of this type, instructions for sample provision by swabbing used toilet paper to collect a small amount of feces (e.g., enough to change colour of or discolour the swab). Therefore, in some preferred embodiments, the sample from the subject is a fecal sample.

In some embodiments, samples can be obtained from the bodies of subjects without facilitation by another entity (e.g., a caretaker associated with a subject, a health care professional, an automated or semi-automated sample collection apparatus, etc.), or can alternatively be taken from bodies of subjects with the assistance of another entity. In some examples, wherein samples are taken from the bodies of subjects without facilitation by another entity in the sample extraction process, a sampling kit can be provided to a subject. In such examples, the kit may include one or more sample collection devices for sample acquisition, one or more containers configured to receive the swab(s) for storage, instructions for sample provision and set-up of a user account, elements configured to associate the sample(s) with the subject (e.g., barcode identifiers, tags, etc.), and a receptacle that allows the sample(s) from the subject to be delivered to a sample processing operation (e.g., by a mail delivery system). In another example, wherein the samples are extracted from the user with the help of another entity, one or more samples can be collected in a clinical or research setting from a subject (e.g., during a clinical appointment).

In some embodiments, a plurality of samples are received from one or more subjects.

Typically, a sample container with the sample from the collection site of the subject is received at the sample handling facility, which functions to enable generation of data from which microbiome-based insights for a subject and/or for a population of subjects can be derived. As noted above, reception of sample containers can be facilitated using one or more of a parcel delivery service and a courier service, or can alternatively be directly enabled with delivery of a sample container to the sample handling facility by the subject associated with the sample container. Preferably, samples received by the sample handling facility are dried due to the sample drying agent included in the sample container.

In some preferred embodiments, an aggregate set of samples is received from a wide variety of subjects, using an aggregated set of sampling kits provided to the subjects by way of the sample handling facility. Preferably, the wide variety of subjects includes subjects of one or more of: different demographics (e.g., genders, ages, marital statuses, ethnicities, nationalities, socioeconomic statuses, sexual orientations, etc.), different health conditions (e.g., health and disease states (including mental health status)), different living situations (e.g., living alone, living with pets, living with a partner, living with children, etc.), different dietary habits (e.g., omnivorous, vegetarian, vegan, sugar consumption, acid consumption, gluten consumption, lactose-free, dairy-free, etc.), different behavioural tendencies (e.g., levels of physical activity, drug use, alcohol use, etc.), different levels of mobility (e.g., related to distance travelled within a given time period), different medication regimens, and any other suitable trait that has an effect on microbiome composition. As such, as the number of subjects increases, the power of insights generated in subsequent blocks of the method increases, in relation to characterizing of a variety of subjects based upon their microbiomes. In some of the same embodiments and other embodiments, the samples received can include receiving biological samples from a targeted group of similar subjects in one or more of: demographic traits health conditions, living situations, dietary habits, behaviour tendencies, levels of mobility, and any other suitable trait that has an effect on microbiome composition, such that insights generated in subsequent steps of the method are insights targeted to specific groups of subjects. Preferably, the set of subjects from which samples are received includes subjects who do not have specific research training, clinical training and/or laboratory training, such that the samples also represent non-trained subjects, who have been instructed in methods of providing samples in a dependable manner.

In some other embodiments, reception of sample containers with samples can be facilitated using a laboratory-based or a clinical-based intermediary that has staff trained in sample extraction from a subject and transmission of extracted samples to the sample sequencing facility. However, reception of the sample at the sample sequencing facility can be enabled in any other suitable manner.

### 4. Sample Processing and Nucleic Acid Sequencing

The methods of the present invention generally include a step of generating a microbiome sequence dataset based upon sequencing nucleic acid content from a microorganism portion of the sample. In this regard, each sample received is processed to determine microbiome composition aspects at the level of a subject and/or the level of a population of subjects. Microbiome composition aspects can include compositional aspects at the microorganism level, including parameters related to distribution of microorganisms across different taxonomic groups of phyla, classes, orders, families, genera, species and/or strain (e.g., as measured in total abundance of each group, relative abundance of each group, total number of groups represented, etc.). In some of the same embodiments and other embodiments the methods may include compositional aspects at the genetic level. Outputs of such sequencing can thus be used to identify features of interest which can be used to characterize the microbiomes of subject and populations of subjects, wherein the features can be microorganism-based (e.g., presence of a genus of bacteria), genetic based (e.g., based upon representation of specific genetic regions and/or sequences), function-based (e.g., based upon representation of specific gene pathways) and/or based on any other suitable scale.

Characterising the microbiome composition associated with a. sample generally includes a combination of sample processing techniques (e.g., wet laboratory techniques) and computational techniques (e.g., bioinformatics) to quantitatively and/or qualitatively characterize the microbiome associated with a sample from a subject.

In some embodiments, sample processing can include any one or more of: lysing a sample; disrupting cell membranes; separation of undesired elements (e.g., proteins) from the sample; purification of nucleic acids (e.g., DNA, RNA) in the sample to generate a nucleic acid sample comprising nucleic acid material derived from a microbiome of the subject and nucleic acid material of the subject; amplification of nucleic acid material of the nucleic acid sample; and sequencing of the amplified nucleic acids of the nucleic acid sample.

In some embodiments, methods of lysing the sample and/or disrupting cell membranes of the sample preferably include physical methods (e.g., bead beating, nitrogen decompression, homogenization, sonication) of cell lysing/membrane disruption, which omit certain reagents that produce bias in representation of certain microorganism groups upon sequencing. In some of the same embodiments and other embodiments, lysing or disrupting membranes can involve chemical methods (e.g., using a detergent, using a solvent, using a surfactant, etc.). In some embodiments separation of undesired elements from the sample can include removal of nucleic acids using nucleases and/or removal of proteins using proteases. In variations, purification of nucleic acids in a sample to generate a nucleic acid sample can include one or more of: precipitation of nucleic acids from the biological samples (e.g., using alcohol-based precipitation methods); liquid-liquid based purification techniques (e.g., phenol-chloroform extraction); chromatography-based purification techniques (e.g., column adsorption); purification techniques involving use of binding moiety-bound particles (e.g., magnetic beads, buoyant beads, beads with size distributions, ultrasonically responsive beads, etc.) configured to bind nucleic acids and configured to release nucleic acids in the presence of an elution environment (e.g., having an elution solution, providing a pH shift, providing a temperature shift, etc.); and any other suitable purification techniques. In some embodiments, the nucleic acid isolation and/or purification is performed using the QIAGEN QIAamp kit.

Technologies for enriching rare sequences in nucleic acid libraries. In some embodiments, methods commonly referred to as Finding Low Abundance Sequences by Hybridization, or “FLASH” is preformed prior to sequencing. FLASH methods use sequence-specific nucleases, such as CRISPR/Cas9, to cut specific sites of interest in a DNA library or other sample prior to sequencing. One advantage of such methods is that it enables enriching for low abundance sequences. FLASH methods are described in detail in the International PCT Patent Publication No. WO2018/035062, which is incorporated herein by reference in its entireity.

In some of the same embodiments and other embodiments nucleic acid fragmentation may be performed using standard techniques in the art (e.g. mechanical fragmentation, enzymatic fragmentation).

In variations, amplification of nucleic acids from the nucleic acid sample preferably includes one or more of: polymerase chain reaction (PCR)-based techniques (e.g., solid-phase PCR, RT-PCR, qPCR, multiplex PCR, touch-down PCR, nanoPCR, nested PCR, hot start PCR, etc.), helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), and any other suitable amplification techniques. In amplification of purified nucleic acids, the primers used may be selected to prevent or minimize amplification bias, as well as configured to amplify nucleic acid regions/sequences that are informative taxonomically and phylogenetically. Thus, universal primers configured to avoid amplification bias can be used in amplification. In some of the same embodiments and other embodiments, primers incorporated barcode sequences specific to each biological sample, as described in further detail below, which can facilitate identification of biological samples post-amplification. Primers used in some embodiments can additionally or alternatively include adaptor regions configured to cooperate with sequencing techniques involving complementary adaptors (e.g., Illumina sequencing). In some embodiments, the primers used can additionally or alternatively be configured to target stable nucleic acid regions (e.g., conserved regions) flanking one or more unstable regions (e.g., mutation-prone regions). Primers used in amplification can, however be configured in any other suitable alternative manner.

In some specific embodiments, amplification and sequencing of nucleic acids from a sample includes: solid-phase PCR involving bridge amplification of DNA fragments of the biological samples on a substrate with oligo adapters, wherein amplification involves primers having a forward index sequence (e.g., corresponding to an Illumina forward index sequence for MiSeq/HiSeq platforms), a forward barcode sequence, a transposase sequence (e.g., corresponding to a transposase binding site for MiSeq/HiSeq platforms), a linker (e.g., a zero, one, or two-base fragment configured to reduce homogeneity and improve sequence results), an additional random base, a sequence for targeting a pre-defined region, a reverse index sequence (e.g., corresponding to an Illumina reverse index for MiSeq/HiSeq platforms), and a reverse barcode sequence. In examples of this type, the sequencing methods comprise Illumina sequencing (e.g., with a HiSeq platform, and/or with a MiSeq platform) using a sequencing-by-synthesis technique.

In some embodiments, the nucleic acid amplification is performed by isothermic amplification. In some of the same embodiments and some other embodiments, the sequencing is performed using the Illumina NovaSeq platform.

In some of the same embodiments and other embodiments, whole genome sequencing methods that randomly sequence DNA fragments in a sample can be used.

### 5. Microbiome Characterisation

The methods of the present invention typically include the step of a processing system, identifying a set of nucleic acids represented in the microorganism portion of the sample, based upon performance of a mapping operation on portions of the microbiome sequence dataset. Computational processing techniques are implemented to transform an input of unanalyzed microbiome sequence data into an output that characterizes represented microorganisms within the sample. Outputs can thus be used to derive values of parameters relative to the relative distributions of microorganism groups within the microbiome of a subject, abundances of microorganism groups within the microbiome of a subject, represented genetic markers within the microbiome of a subject and/or any other suitable parameters, as further described below. In some embodiments, computational processing can include any one or more of: identifying sequences associated with the microorganism portion (as opposed to human sequences and contaminants), and performing alignment and mapping of sequences associated with the microorganism portion (e.g., alignment of fragmented sequences using one or more of single ended alignment, ungapped alignment, gapped alignment, pairing).

Identifying sequences associated with the microorganism portion, can include mapping of sequence data from sample processing to a human reference genome (e.g., provided by the Genome Reference Consortium), in order to remove human genome-derived sequences. Additionally, identifying sequences associated with the microorganism portion can include discarding sequences associated with unintelligible and/or low quality reads at a module of the processing system configured to perform quality filtering of reads (e.g., according to the use of Q or Phred quality scores), such that only non-human and high quality reads (e.g., reads above a certain quality score threshold in terms of a Q or Phread score) remain. However, identifying sequences associated with the microorganism portion can be performed in any other suitable manner.

Any unidentified sequences remaining after mapping of sequence data to the human reference genome can then be further clustered into operational taxonomic units (OTUs) based upon sequence similarity and/or reference-based approaches (e.g., using VAMPS, using MG-RAST, using QIIME databases), assembled based upon overlapping with other reads, and aligned to reference sequences. Alignments can be performed in multiple phases, using one or more of: single-ended alignment, ungapped alignment, gapped alignment, paired alignment (e.g., with forward and reverse pairs of sequences), and any other suitable phase of alignment. Furthermore, alignment algorithms implemented at the processing system can be configured for specific read lengths of ranges of read lengths, in order to increase the efficiency of alignment processing based upon sequence lengths. Alignment algorithms can implement a hashing approach with large contiguous seeds and/or with adaptive stopping techniques whereby a read is considered to be aligned based upon a determination of the best read alignment across a set of read alignment candidates, and the number of read alignment candidates considered. Alignment algorithms can additionally or alternatively include string comparison algorithms that compare a number of mismatches between two strings (e.g., a reference read and a sequence read) of the same length. Furthermore, in some embodiments alignment algorithms can use profile stochastic contest-free grammars (e.g., implementing covariance models), using, for example, an SSU-align algorithm. Any other suitable type of alignment algorithm can be used.

In some embodiments, alignment and mapping to reference bacterial genomes (e.g., provided by the National Center for Biotechnology Information) can be performed using an alignment algorithm that performs a global alignment of two reads (e.g., a sequencing read and a reference read) with a stopping condition based upon scoring of the global alignment (e.g., in terms of insertions, deletions, matches, mismatches); a Smith-Waterman algorithm that performs a local alignment of two reads (e.g., a sequencing read and a reference read) with scoring of the global alignment of two reads (e.g., a sequencing read and a reference read) with scoring of the local alignment (e.g., in terms of insertions, deletions, matches, mismatches); a Basic Local Alignment Search Tool (BLAST) that identifies regions of local similarity between sequences (e.g., a sequencing read and a reference read)' a FPGA accelerated alignment tool; a BWT-indexing with BWA tool; a BWT-indexing with SOAP tool; a BWT-indexing with Bowtie tool; Sequence Search and Alignment by Hashing Algorithm (SSAHA2) that maps nucleic acid sequencing reads onto a genomic reference sequencing using work hashing and dynamic programming; and any other suitable alignment algorithm. Mapping of unidentified sequences can further include mapping to reference viral genomes, fungal genomes and/or parasitic genomes, in order to further identify viral and/or fungal components of the microbiome of a subject. Furthermore, overlapping reads (e.g., generated by paired end sequencing) can be assembled based upon outputs of the alignment algorithm or aligned sequence reads can be merged with reference sequences (e.g., using a hidden Markov model banding technique, using a Durbin-Holmes technique). Alignment and mapping can, however, implement any other suitable algorithm or technique.

Mapping of encoded sequence to reference sequences can, however, be performed in any other suitable manner.

The processing system is suitably in direct communication with the sequencing facility. In some embodiments, the sequencing facility can be configured to provide sequenced data as an output to a module of the processing system. In some of the same embodiments and some other embodiments, the processing system can be configured to receive inputs from outputs of the sample sequencing facility. The processing system is preferably implemented in one or more computing systems, wherein the computing system(s) can be implemented at least in part in the cloud and/or as a machine (e.g., computing machine, server, etc.) configured to receive a computer-readable medium storing computer readable instructions. In some embodiments of this type, the processing system can comprise one or more processing modules, implemented in the cloud and/or as machine, comprising instructions for performing blocks of the method described above and/or elsewhere herein. By way of an illustrative example, the processing system can include a first module configured to receive data derived from outputs of the sequencing facility, a second module configured to align and map sequenced data from the first module as described above, and a third module configured to receive outputs of the second module in order to generate features and derive insights, as described, below.

Sample identification In some embodiments, the disclosed methods of processing a sample to generate a microbiome sequence dataset from a sample include an identification step that combines one or more nucleic acid index sequences within each sample or for each individual associated with a set of samples received at the sample sequencing facility. Use of index sequences can thus function to enable identification of samples in association with a specific individual, enable detection of contamination (e.g., cross-contamination) of samples, and facilitate quantification of reads associated with given sequences in a sample that is processed in a multiplexed manner.

As noted above, the index sequences can be associated with primers implemented during an amplification process, or otherwise combined with a sample in any other suitable manner.

### 6. Microbiome Insights

Another step generally applied to the methods of the present application include generating an analysis based upon a set of features related to the microorganism portion of the sample. Such analysis typically functions to transform outputs into features that can be processed algorithmically to determine microbiome-based insights at the subject level and the population of subjects level. This can include generating an analysis based upon features derived from compositional aspect of the microbiome associated with the sample.

Upon identification of represented groups of microorganisms of the microbiome associated with a sample, based upon the mapping and alignment operations described above, generating features derived from compositional aspects of the microbiome associated with a sample can be performed. In some embodiments, generating features can include generating features that describe the presence or absence of certain taxonomic groups of microorganisms and/or the relative abundance of specific microorganism species or strains. In some of the same embodiments and other embodiments, generating features can include inferring phylogenetic traits associated with aligned, mapped, and/or merged reads, which can include determining placement of sequences on a reference phylogenetic tree of microorganisms. In some of the same embodiments and other embodiments, generating features can include generating features describing quantities of represented taxonomic groups. Additionally, or alternatively, generating features can also include generating features describing diversity of different microorganism groups and relative abundance of different microorganism groups. Generating features may include generating features describing diversity of different microorganism groups and relative abundance of different microorganism groups and relative abundance of different microorganism groups, for instance, using a Genome Relative Abundance and Average size (GAAS) approach and/or a Genome Relative Abundance using Mixture Model theory (GRAMMy) approach that uses sequence-similarity data to perform a maximum likelihood estimation of the relative abundance of one or more groups of microorganism. In some of the same embodiments and other embodiments, generating features can include generating statistical measures of taxonomic variation, as derived from abundance metrics. Additionally, or alternatively, generating features can include generation of qualitative features describing presence of one or more taxonomic groups, in isolation and/or in combination. Additionally, or alternatively, generating features can include generation of features related to genetic markers characterizing microorganism of the microbiome associated with a biological sample.

In some of the same embodiments and some alternative embodiments, generating features can include quantification of abundance information regarding the potential capacity of a microorganism, or a community of microorganisms, to perform a specific metabolic function, or a group of metabolic functions. The abundance information may be relative to other microorganisms, or relative to other communities of microorganisms.

### 7. Supplementary Information

Upon feature generation, generating an analysis based upon the generation of features may be performed. In generating of the analysis, supplementary data may be implemented that can enhance correlations and/or predictions included in the analysis. Accordingly, in some embodiments the method further comprises receiving a supplementary dataset that includes demographic and behavioural information from at least one of the subject and the population of subjects. The supplementary dataset preferably includes survey-derived data. However, in some embodiments of this type, the supplementary data may additionally or alternatively include any one or more of: contextual data derived from sensors, medical data, and any other suitable type of data (e.g., blood tests, metabolic analysis, human DNA test, etc.).

In some embodiments, the reception of supplementary data includes the reception of survey-derived data. Preferably, the survey-derived data preferably provides physiological, demographic, and behavioural information in association with a subject. Physiological information can include information related to physiological features (e.g., height, weight, body mass index, body fat percent, body hair level, etc.). Demographic information can include information related to demographic features (e.g., gender, age, ethnicity, marital status, number of siblings, socioeconomic status, sexual orientation, etc.). Behavioural information can include information related to one or more of: health conditions (e.g., health and disease states, including but not limited to mental health status); living situations (e.g., living alone, living with pets; living with a partner; living with children, etc.); dietary habits (e.g., omnivorous, vegetarian, vegan, sugar consumption, acid consumption, fibre consumption, fat consumption, etc.); behavioural tendencies (e.g., levels of physical exercise, drug use, alcohol use, etc.); different levels of mobility (e.g., related to distance travelled within a given time period); different levels of sexual activity (e.g., related to numbers of partners and sexual orientation); and any other suitable behavioural information. In one example, a survey configured to facilitate generation of the supplementary dataset includes a question related to height of the subject, weight of the subject, diet of the subject, alcohol consumption of the subject, and diet beverage consumption. Survey-derived data can thus include quantitative data and/or qualitative data (e.g., using scales of severity, mapping of qualitative response to quantified score, etc.).

In facilitating reception of survey-derived data, one can include providing one or more surveys to a subject, or to an entity (e.g., healthcare provider, care-taker, spouse, relative, etc.) associated with the subject. Survey data can be provided in person (e.g., in coordination with sample provision and reception from a subject), electronically (e.g., during account setup by a subject, at an application executed at an electronic device of a subject), and/or in any other suitable manner.

In some of the same embodiments and other embodiments, portions of the supplementary dataset can be derived from sensors associated with the subjects (e.g., sensors on wearable computing devices, sensors on mobile devices, biometric sensors associated with the user, etc.). The provision of this data can include receiving one or more of: physical activity or physical action-related data (e.g., accelerometer and gyroscope data from a mobile device or wearable electronic device of a subject); environmental data (e.g., temperature data, elevation data, climate data, light parameter data, etc.); patient nutrition or diet-related data (e.g., data from food establishment check-ins, data from spectrophotometric analysis, etc.); biometric data (e.g., data recorded through sensors within the patient's mobile computing device, data recorded through a wearable or other peripheral device in communication with the patient's mobile computing device, location area (e.g., using GPS elements); and any other suitable data. In some of the same embodiments and other embodiments, generation of association between features (or values of parameters derived from features) and information derived from the supplementary dataset, generation of confidence metrics or measures of correlational strength between microbiome-based features (or values of parameters derived from features) and behavioural or demographic characterization derived from the supplementary data and any other suitable insights. In some embodiments, portions of the analysis can support or provide diagnostic tools that can characterize a subject (e.g., in terms of behavioural trait, in terms of medical conditions, in terms of demographic traits, etc.) based upon their microbiome composition, and/or predict a subject's microbiome composition, and/or predict a subject's microbiome composition based upon one or more of their behavioural traits, medical conditions, demographic traits and any other suitable traits.

Portions of an analysis can be derived from machine learning-based techniques, whereby input data derived from generated features can be processed with a training dataset having features like to candidate classification, e.g., derived from a supplementary dataset) to provide a classification model, microbiome based features (or values of parameters derived from features) and behavioral or demographic characteristics derived from the supplementary dataset, and/or any other suitable insights. In some embodiments, portions of the analysis can support or provide diagnostic tools that can characterize a subject (e.g., in terms of behavioural traits, in terms of medical conditions, in terms of demographic traits, etc.) based upon their microbiome composition, and/or predict a subjects' microbiome composition based upon one or more of their behavioural traits, medical conditions, demographic traits, and any other suitable traits.

Portions of an analysis can be derived from machine learning-based techniques, whereby input data derived from generated features can be processed with a training dataset having features linked to candidate classifications (e.g., derived from a supplementary dataset) to provide a classification model that links microbiome-based features to other characteristics of a subject. In some embodiments, a classification model can be trained to identify microbiome-based features and/or feature combinations that have high degrees (or low degrees) of predictive power in accurately predicting a classification of a subject. As such, refinement of the classification model with the training dataset identifies feature sets (e.g., of individual features, of combinations of features) having high correlation with specific classifications of subjects.

Feature selection approaches can include correlation feature selection (CFS) methods, consistency methods, relief methods, information gain methods, symmetrical uncertainty methods, and/or any other suitable methods of feature selection. In one variation, the feature vectors can include features related to one or more of: microbiome diversity metrics (e.g., in relation to distribution across taxonomic group, in relation to distribution across bacterial, viral, and/or fungal groups), presence of taxonomic groups in one's microbiome, representation of specific genetic sequences in one's microbiome, microbiome resilience metrics (e.g., in response to a perturbation determined from the supplementary dataset), and any other suitable features derived from the microbiome diversity dataset and/or the supplementary dataset. Additionally, combinations of features can be used in a feature vector, wherein features can be groups and/or weighted in providing combined features as part of a feature set.

In some embodiments, the generation of a classification molecule is performed using a machine-learning classifier, the classification model can be generated and trained according to a random forest predictor (RFP) algorithm that combines bagging (i.e., bootstrap aggregation) and selection of random sets of features from a training dataset to construct a set of decision trees, T, associated with the random sets of features. In using a random forest algorithm, N cases from the set of decision trees are sampled at random, with replacement to create a subset of decision trees, and for each node, m prediction features are selected form all of the prediction features for assessment. The prediction feature that provides the best split at the node (e.g., according to an objective function) is used to perform the split (e.g., as a bifurcation at the node, as a trifurcation at the node). By sampling many times from a large dataset, the strength of the classification molecule, in identifying features that are strong in predicting classifications can be increased substantially. In this embodiment, manure to prevent bias, (e.g., sampling bias) and/or account for an amount of bias can be included during processing to increase robust of the model.

While a random forest method of machine learning is described in the variation above, any other suitable machine learning algorithm is equally as applicable in forming and/or training the classification model. In some embodiments, the machine learning algorithm(s) can be characterized by a learning style including any one or more of: supervised learning (e.g., using logistic regression, using back propagation neural networks), unsupervised learning (e.g., using an Apriori algorithm, using K-means clustering), semi-supervised learning, reinforcement learning (e.g., using a Q-learning algorithm, using temporal difference learning), and any other suitable learning style. Furthermore, the machine learning algorithm can implement any one or more of: a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averages one-dependence estimators, Bayesian believe footwork, etc.), a kernel method (e.g., a support vector machine, a radial basis function, a linear discriminate analysis, etc.), a clustering method (e.g., k-means clustering, expectation maximization, etc.), an associated rule learning algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine; a deep believe network method, a convolution network method, a stacked auto-encoder method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and any suitable form of machine learning algorithm, some forms of which are described in U.S. Patent Application No. 61/953,683, entitled “Multiplex Markers” and filed on 14 May 2014.

In some of the same embodiments and other embodiments, portions of the analysis can be generated using statistical methods and tools, including one or more of: basic statistics, scatterplot analysis, principal component analysis (PCA), edge PCT, UniFrac analyses (e.g., to calculate distances between identified microorganism communities using phylogenetic information), multivariate analyses, analyses of variance, cluster analyses, Kantorovich-Rubinstein metrics, and any other suitable statistical method.

The methods of the present invention also comprise the step of transmitting information derived from values of the set of parameters to the subject, which functions to share insights derived from the analysis described above and elsewhere herein, with one or more subjects. Transmitting information to a subject can be facilitated by way of the user account for the subject, set up as described above, such that the information is accessible at an electronic device (e.g., personal computer, smart phone, head-mounted wearable computing device, wrist-mounted wearable computing device, tablet, laptop, notebook, etc.) of the subject. Additionally, or alternatively, information can be provided to the subject in the form of a printed report, an electronic document (e.g., a PDF), as raw data, and/or in any other suitable form.

In some embodiments, the information can indicate one or more of: the presence of one or more microorganisms in a subject's microbiome, the absence of one or more microorganisms in an subject's microbiome; the abundance (e.g., relative abundance or absolute abundance) of one or more microorganisms in a subject's microbiome; and comparisons between the microbiome composition of a subject relative to one or more subpopulations of subjects or populations of subjects based upon any physiological demographic, or behavioural classification. Information can suitably be provided in the context of average, typical or healthy ranges. In some embodiments, the information provided to a subject can depict an amount of a given type of microorganism present in a sample from a subject with reference to an average range of amounts of the given type of microorganism and reference to a full range of amounts for the given type of microorganism from a population of subjects.

Information provided can be organized into different use levels, wherein each user level can have access to different data, analyses and/or other tools. For instance, user levels can be organized according to one or more of profession (e.g., scientist, researcher, clinician, healthcare provide, etc.), status (e.g., consumer, patient), and any other classification of user level. For instance, in one example, scientists/researchers can be permitted to upload research or study data, compare research or study data to other research of study data, compare research or study data from different subpopulations of subjects, and predict results of a larger study from results of a pilot study. In another example, clinicians can be permitted to view patient information, and patients can be permitted to share information with their clinicians.

Information can be provided (e.g., in an electronic report, a printed report, etc.) or rendered at an electronic display using visualization tools for taxonomic data (e.g., graphics and/or tables showing domain, kingdom, phylum, class, order, family, genus, species, subspecies and/or strain relationships), phylogenetic trees, cladograms, dendrograms, pie charts, bar charts, scatter plots, treeplots and any other suitable visualization tool. Furthermore, a user interface associated with a user account can provide controls, to adjust levels of detail provided to the subject, to adjust types of comparison information provided to the subject, and/or to adjust any other suitable parameter pertaining to information provided to the subject.

Information provided can be rendered at a display in any suitable form including (but not limited to) one or more of: a scatterplot, a network chart, a pie chart, a table, a treemap, a set of comparison diagrams between microbiome compositional features of a subject in comparison to one or more subpopulations of subjects, and a set of comparison matrices between microbiome compositions features of a subject in comparison to one or more subpopulations of subjects. In one example, the graphical representations may include rendering a chart displaying microbiome compositional information for a sample from a subject, with a legend describing represented microbiome components. In another example, the graphical representation may include rendering a set of charts comparing the microbiome composition of a sample from a subject to an average of all samples provided from a subject to an average of all samples provided from a population of subjects at a taxonomic level (e.g., genus level), in coordination with a user interface that allows a subject to receive information at other taxonomic levels (e.g., the domain level, the phylum level, the class level, the order level, the family level, the genus level, the species level, the sub-species level) upon receiving of an input at the user interface by the subject. In yet another example, the graphical representations can include comparing the microbiome composition of a sample from a subject to the average microbiome compositions for a subpopulation of healthy omnivores, the average microbiome composition for a subpopulation of vegetarians, and the average microbiome composition for the entire population of subjects analysed.

### 8. Best Method

In some embodiments, the method of the invention comprises a workflow 100 in which a subject receives a sampling kit 110, interacts with the sampling kit 115, and provides samples for analysis by using components of the sampling kit. In the workflow, the sample(s) from a subject is received 120, processed 130, analyzed 140, and used to provide information to the subject 160.

A subject receives a sampling kit 110, transmits one or more samples from one or more collection sites into sample containers of the sampling kit 115, and returns the sample containers to a sample handling facility by way of packaging receptacles included in the sampling kit, 120. Registration codes (e.g., barcodes) associated with the sampling kit and the sample collection container(s) are logged, at the sample handling facility, for tracking. Samples from the subject are then introduced into an automated sample handling workflow implementing a sequencing facility and a processing system, wherein nucleic acids from the samples are purified, amplified, tagged, and sequenced 140. Data derived from sequenced nucleic acids are then associated with samples based upon identifiers (e.g., index sequences, tags, etc.) and analysed to derive microbiome information 150. Information pertaining to the microbiome of the subject is then presented to the subject by way of an interactive website that provides renderings of graphs, charts, and comparisons between the microbiome of each sample from the subject, and relevant subpopulations of subjects, relevant ranges of metrics, and/or relevant microbiome-based studies 160.

The method and/or system of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a patient or computer or mobile device, or any suitable combination thereof. Other systems and methods of the embodiments can be embodied and/or implemented at least in part as a machine configured to receive a computer-readable medium storing computer readable instructions. The instructions can be executed by computer-executable components integrated by computer executable components integrated with apparatuses and networks of the types described above. The computer readable medium can be stored on any suitable computer readable media such as RAMs, ROMs, flash memory, EEPROMs, optical decides (CD or DVD), hard drives, floppy drives, or any suitable device. The computer-executable component can be a processor, though any suitable dedicated hardware device can (alternatively or additionally execute the instructions.

In some of the same embodiments and other embodiments, the methods include the additional step of Depletion of Abundant Sequences by Hybridization (“DASH”). In embodiments of this type, sequencing libraries can be “DASHed” with recombinant Cas9 protein complexed with a library of guide RNAs targeting unwanted species for cleavage, thus preventing them from consuming sequencing space. Suitable DASH methods that can be used in the methods of the present invention are described in the art, including in U.S. Patent Publication No. 2018/0051320, which is incorporated herein by reference in its entireity.

### EXAMPLES

### Example 1

### Intra-Stabilization of Community Diversity

In order to assess the sample stabilising properties of the proposed sample collection device and extraction methods, the inventors performed a comparative study with the commonly used sample stabilisation techniques and remote sample testing products.

Replicate samples were sequenced from five subjects after storing for four weeks with six sample stabilization techniques:

Swab with no lysis/processing buffer, and with an active drying system (“Dry Swab”);

### BBL CultureSwab EZ (BD Diagnostics);

### OMNIgene-GUT (DNA Genotek);

### RNAlater Stabilization Solution (Thermo Fisher Scientific);

### LifeGuard Preservation Solution (Qiagen); and

### DNA/RNA Sheild (Zymo Research).

Samples from each subject were also frozen immediately as control time zero baselines. Species profiles for all samples were obtained with the Microba Community Profiler (MCP v1) data processing after removing poor-quality and human-associated reads and subsampling to 7 million pairs. These profiles were then compared to determine the community stability for each of the above-listed stabilisation conditions.

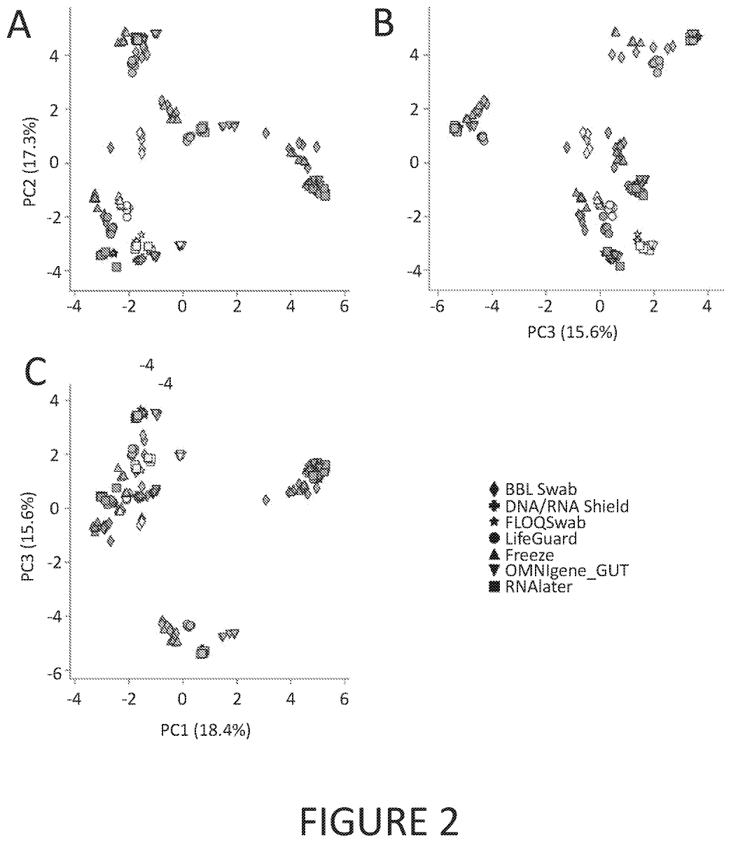

An overview of all samples is provided as a principal component analysis plot of Hellinger transformed species profiles (FIG. 2). Samples cluster to a large degree by subject, though there is not clear separation between subjects 2 and 4. Surprisingly, the different swab stabilization techniques have a notable impact on the species profiles. Notably, the BBL swab samples from subject 2 appear to be outliers.

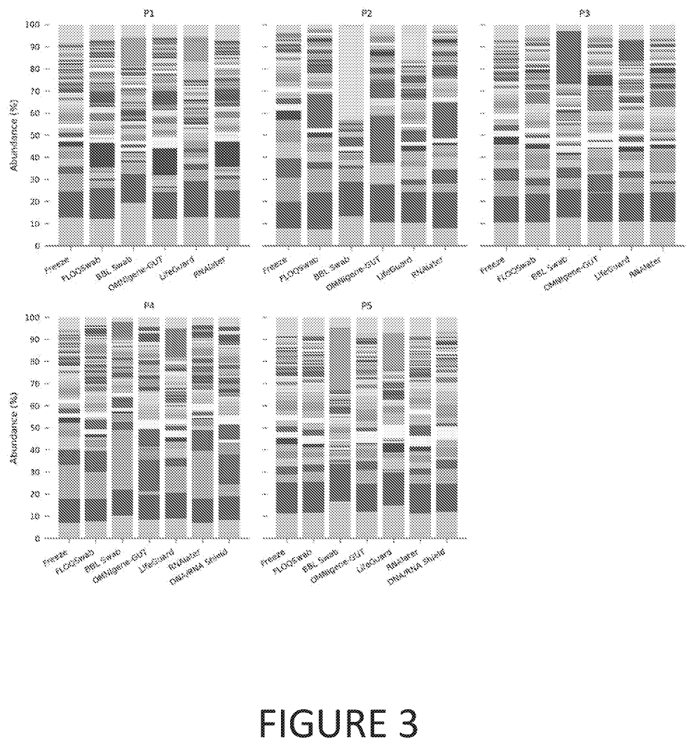

Species profiles averaged over the six replicates were determined for each subject and stabilization technique (FIG. 3). Substantial blooms relative to the baseline “freeze” samples are noticable. The highly abundant populations in the BBL Swab samples that are not observed in the baseline samples are E. coli for subjects 1 to 4 and Escherichia sp2 for subject 5.

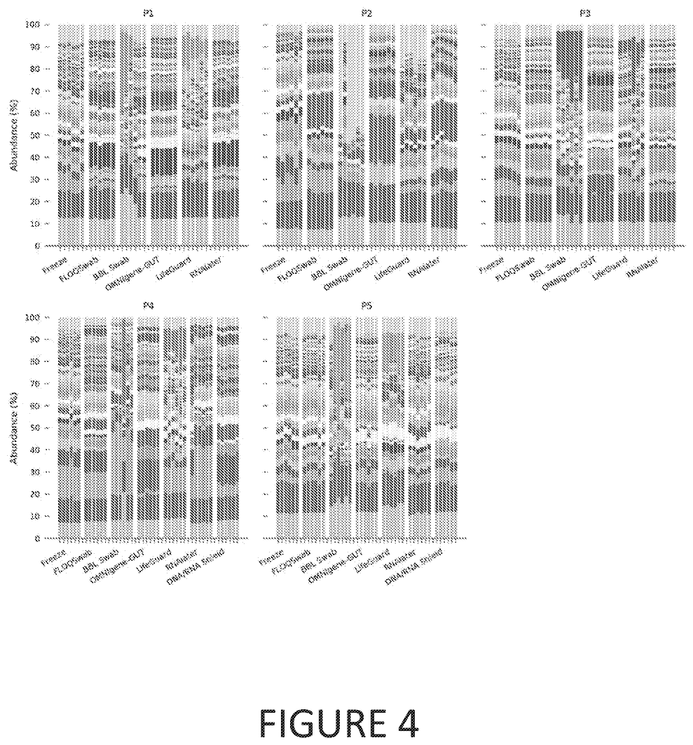

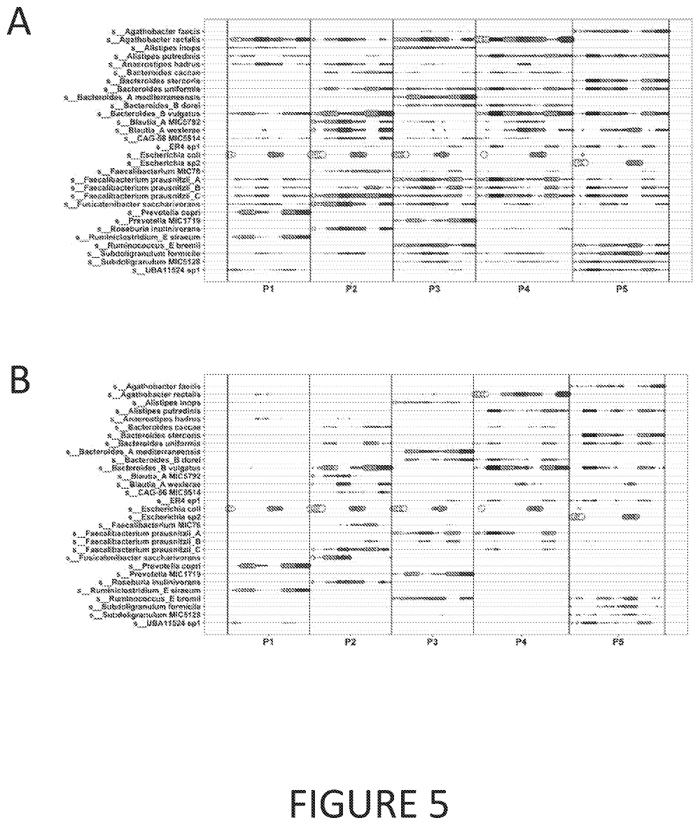

Individual species profiles show reasonable variability between replicates (FIGS. 4 and 5). Substantial variability can be observed between replicates in some cases. In particular, the E. coli outliers observed for the BBL Swab samples are not present in all replicates.

Beta diversity was measured with the following analyses:

Bray-Curtis—which considers the abundance of individual species;
Hamming and Sorensen—which considers only the presence/absence of species. Hamming distance is the number of species that differ between two samples.
Sorensen normalizes the Hamming distance to account for how many species are contained in the two samples.

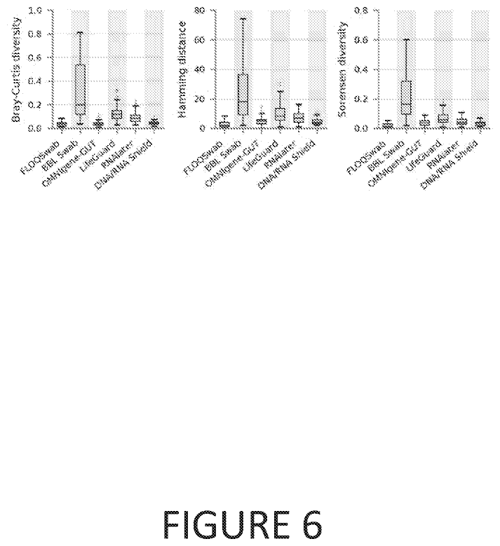

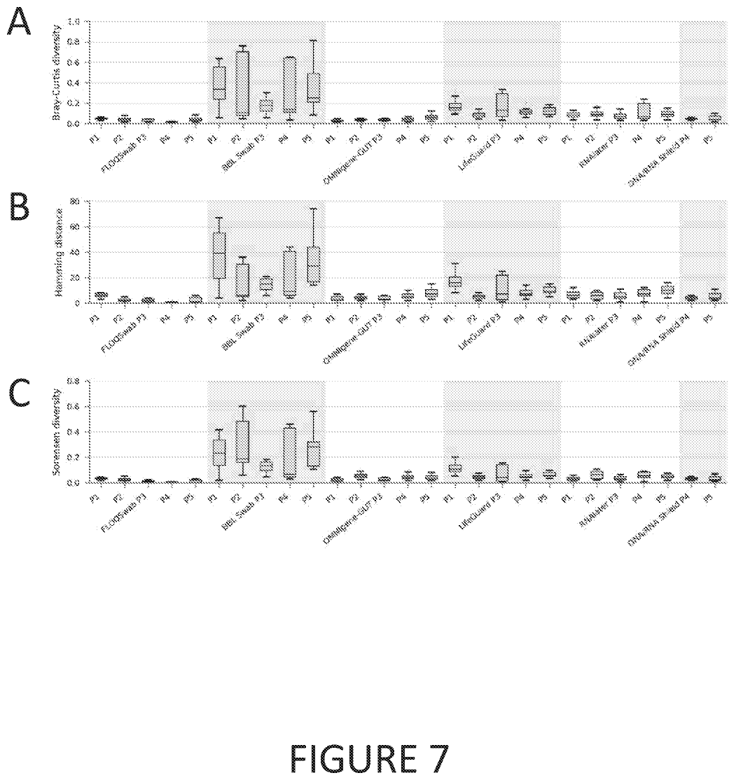

Aggregated results for all samples using a specific stabilization technique are given in FIG. 6. Results broken out by individual are shown in FIG. 7.

The community profiles obtained with the BBL CultureSwab technique shows extremely high diversity (FIG. 6) for several individuals (FIG. 7). Examination of the samples confirm that this is not due to a sample labelling issue as samples from each participant generally cluster together.

### Example 2

### Inter-Stabilization Community Diversity

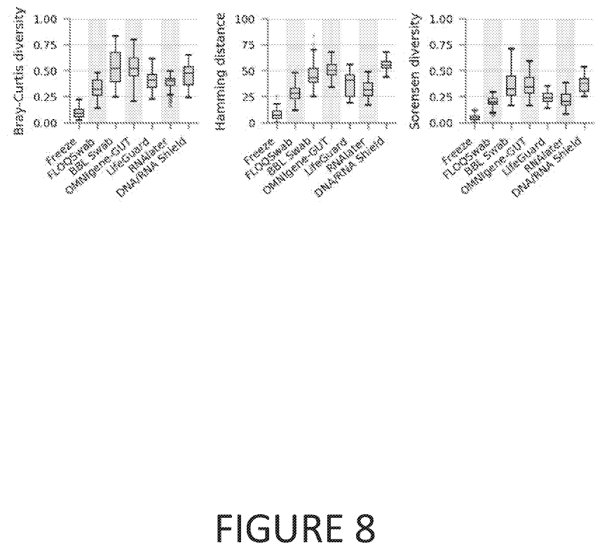

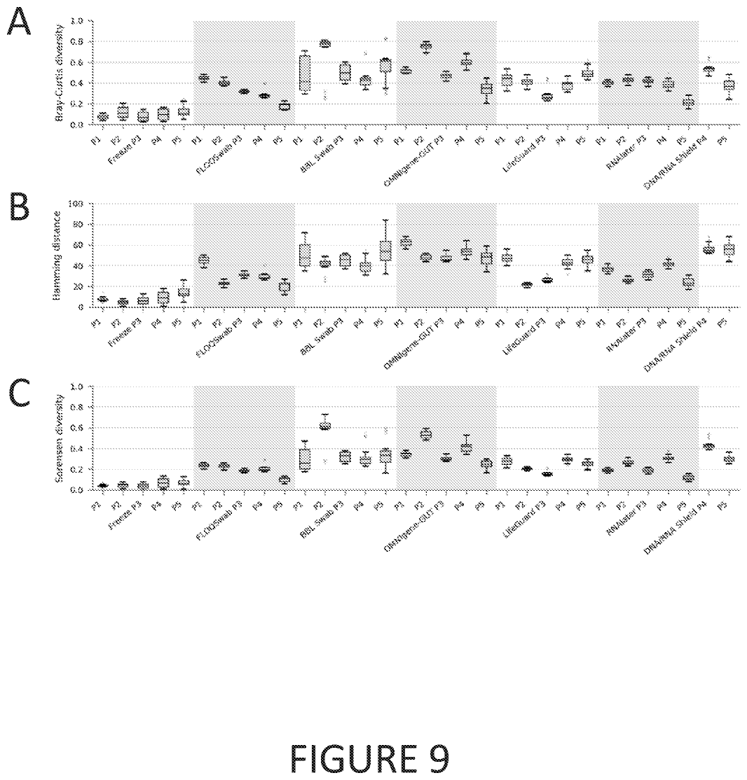

After validating the performance of the swabs comprising no lysis or processing agent/buffer, but rather an active drying agent, as shown above, the inventors performed a comparative study between species profiles under the different stabilization techniques and those obtained at time zero (see, FIGS. 8 and 9). Surprisingly, these results suggest that using a dry swab results in species profiles that are closest to those obtained time zero (see, FIG. 8).

### Materials & Methods

Five participants provided fecal samples of greater than 10 grams (g). Each sample was homogenised and equally divided between the following stabilisation techniques (in triplicate):

Immediate freezing;
RNA Later (0.5 g sample requires around 2.5 mL of RNAlater solution);
LifeGuard (Qiagen) (between 2 and 2.5 volumes of LifeGuard Soil Preservation Solution per gram).

Samples were stored for four weeks before being sequenced.

Stool samples collected from subjects as produced and immediately proceed to sample processing.

At least 10 g of the total stool sample is stored in a sterile container and homogenised by stirring for two minutes with a sterile spatula. Stool sample was then divided into thirty three (33) 100 mg aliquots.

Six aliquots were transferred into 2 mL Eppendorf tube on dry ice and placed immediately into the −20° C. freezer.

Six aliquots were transferred into 2 mL Eppendorf tubes containing 1 mL RNAlater. The samples were left at room temperature for 1 week, before being frozen at −20° C. for three weeks.

Six aliquots were transferred into 2 mL Eppendorf tubes with 1 mL of LifeGuard. The samples were left at room temperature for 1 week, before being frozen at −20° C. for three weeks.

Six aliquots were added to Copan FLOQSwab swabs, and retained at room temperature for the total four weeks.

Six aliquots were added to BD BBL CultureSwab EZ Sterile Swabs. The samples were left at room temperature for 1 week, before being frozen at −20° C. for three weeks.

### REFERENCES

Clarridge, J. E., Impact of 16S rRNA gene sequence analysis for identification of bacteria on clinical microbiology and infectious diseases. Clinical Microbiology Reviews, 17(4), 840-862 (2004).
Huse, S. M., Welch, D. M., Morrison, H. G., Sogin, M. L., Ironing out the wrinkles in the rare biosphere through improved otu clustering. Environmental Microbiology, 12(7), 1889-1898 (2010).

### Peterson, J, and Garges, S., et al., (2009) The NIH Human Microbiome Project, Genome Res., 18 (12): 2317-2323.

Sender R., Fuchs, S., Milo R., (2016) Reviesed estimates for the number of human and bacteria cells in the body, PLOS Biology.

1. Use of a sample collection device in a nucleic acid sequencing process, wherein the sample collection device comprises: (i) a container; (ii) a collection element comprising a support body and a collection portion; and (iii) a sample drying agent.

2. The use of claim 1, wherein the nucleic acid sequencing process comprises whole genome sequencing.

3. Use of a sample collection device in the manufacture of a kit for performing whole genome sequencing on a sample obtained from a subject, wherein the sample comprises nucleic acid material; and wherein the kit comprises: (i) a container; (ii) a collection element comprising a support body and a collection portion; and (iii) a sample drying agent.

4. The use of any one of claims 1 to 3, wherein the nucleic acid material is derived from a microorganism present in a microbiome (e.g., a gut microbiome) of the subject.

5. The use of any one of claims 1 to 4, wherein the support body comprises a longitudinal extension.

6. The use of any one of claims 1 to 5, wherein the sample drying agent is located at least partially within the container.

7. The use of claim 6, wherein the sample drying agent comprises a composition selected from activated alumina, aerogel, benzophenone, bentonite clay, calcium chloride, calcium oxide, calcium sulfate, cobalt(II) chloride, copper(II) sulfate, lithium chloride, lithium bromide, magnesium sulfate, magnesium perchlorate, potassium carbonate, potassium hydroxide, silica, sodium, sodium chlorate, sodium chloride, sodium hydroxide, sodium sulfate, sucrose, and sulfuric acid.

8. The use of claim 7, wherein the sample drying agent is substantially composed of silica.

9. The use of claim 7 or claim 8, wherein the sample drying agent is housed in a sachet or packet.

10. The use of any one of claims 1 to 9, wherein the collection portion comprises a plurality of elongated fibres.

11. The use of claim 10, wherein the elongated fibres are substantially composed of a synthetic or artificial material.

12. The use of claim 11, wherein the synthetic or artificial material is selected from: nylon, rayon, polyester, polyamide, carbon fibre, alginate or a mixture thereof.

13. The use of claim 10, wherein the elongated fibres are substantially composed of a natural material.

14. The use of claim 13, wherein the natural material is selected from: cotton, silk, or a mixture thereof.

15. The use of any one of claims 10 to 14, wherein the elongated fibres have hydrophilic properties.

16. The use of any one of claims 10 to 16, wherein the plurality of fibres is arranged as a layer having a substantially uniform thickness.

17. The use of any one of claims 10 to 16, wherein the fibres are deposited on the collection portion by flocking in an ordered arrangement of the fibres normal to the non-absorbent surface.

18. The use of any one of claims 1 to 17, wherein the sample collection device is configured for collecting a sample selected from the group comprising: a fecal sample, a saliva sample, a skin sample, an oral sample, a genital sample, a nasal sample, an eye sample, and an ear sample.

19. The use of any one of claims 1 to 18, wherein the sample collected using the device is used to characterise a microorganism present in the subject.

20. The use of any one of claims 1 to 19, wherein the sample collected using the device is used to fully or at least partially characterise the subject's microbiome.

21. A method of preparing a nucleic acid sample, the method comprising:
a. providing a sampling kit to a subject at a remote location, wherein the sampling kit comprises: (i) a container, configured to receive a sample from a collection site of the subject; (ii) a collection element comprising a support body and a collection portion; and (iii) a sample drying agent;
b. receiving the sample container with a sample from the subject, wherein the sample is received in a dried state.

22. The method of claim 21, wherein the method further comprises nucleic acid sequencing (e.g., whole genome nucleic acid sequencing).

23. The method of claim 21 or claim 22, wherein the nucleic acid material is derived from at least one microorganism within the sample.

24. The method of claim 23, wherein the sample is selected from the group comprising: a fecal sample, a saliva sample, a skin sample, an oral sample, a genital sample, a nasal sample, an eye sample, and an ear sample.

25. The method of claim 24, wherein the at least one microorganism is present in the gut microbiome of the subject.

26. The use of any one of claims 21 to 25, wherein the support body comprises, consists, or consists essentially of, a longitudinal extension, optionally with a non-absorbent surface.

27. The use of claim 26, wherein the sample drying agent is located at least partially within the container.

28. The use of claim 27, wherein the sample drying agent comprises a composition selected from activated alumina, aerogel, benzophenone, bentonite clay, calcium chloride, calcium oxide, calcium sulfate, cobalt(II) chloride, copper(II) sulfate, lithium chloride, lithium bromide, magnesium sulfate, magnesium perchlorate, potassium carbonate, potassium hydroxide, silica, sodium, sodium chlorate, sodium chloride, sodium hydroxide, sodium sulfate, sucrose, and sulfuric acid.

29. The use of claim 28, wherein the sample drying agent is substantially composed of silca.

30. The use of claim 28 or claim 29, wherein the sample drying agent is housed in a sachet or packet.

31. The use of any one of claims 21 to 30, wherein the collection portion comprises a plurality of elongated fibres.

32. The use of claim 31, wherein the fibres have hydrophilic properties.

33. The use of claim 31 or claim 32, wherein the plurality of fibres are arranged as a layer having a substantially uniform thickness.

34. The use of any one of claims 21 to 33, wherein the fibres are deposited on the tip portion by flocking on an ordered arrangement of the fibres normal to the non-absorbent surface.

35. The use of any one of claims 21 to 34, wherein the sample collection device is configured for collecting a sample from the subject selected from: a fecal sample, a saliva sample, a skin sample, an oral sample, a genital sample, a nasal sample, an eye sample, and an ear sample.

36. The use of any one of claims 21 to 35, wherein the sample collected using the device is used to characterise the gut microbiome of the subject.

37. A method of preparing a sample for nucleic acid sequencing, the method comprising:
a. providing a sampling kit to a subject at a remote location, wherein the sampling kit comprises a sample collection device, the device comprising: (i) a container, configured to receive a sample from a collection site of the subject; (ii) a collection element comprising a support body and a collection portion; and (iii) a sample drying agent;
b. drying the sample with a sample drying agent; and
c. receiving the sample at a sample sequencing facility.

38. A method of preparing a sample for nucleic acid sequencing, the method comprising:
a. providing a sampling kit to a subject at a remote location, wherein the sampling kit comprises a sample collection device, the device comprising: (i) a container, configured to receive a sample from a collection site of the subject; (ii) a collection element comprising a support body and a collection portion; and (iii) a sample drying agent;
b. receiving the sample container with a sample from the subject; and
c. resuspending the sample in a buffer.

39. A method of characterising the composition of a microbiome in a subject, the method comprising:
a. providing a sampling kit to a subject at a remote location, wherein the sampling kit comprises a sample collection device, the device comprising: (i) a container, configured to receive a sample from a collection site of the subject; (ii) a collection element comprising a support body and a collection portion; and (iii) a sample drying agent;
b. receiving the sample container containing a sample from the subject;
c. sequencing nucleic acid material from the microorganism portion of the sample, to generate a microbiome sequence dataset;
d. identifying a set of microorganisms present in the microorganism portion of the sample, based on the microbiome sequence dataset;
e. generating an analysis based on the set of microorganisms present in the microorganism portion of the sample; and
f. communicating the analysis to the subject.

40. The method of claim 39, wherein the microbiome is a gut microbiome.

41. The method of any one of claims 37 to 40, wherein the nucleic acid sequencing is whole genome nucleic acid sequencing.

42. The method of any one of claims 37 to 41, wherein the nucleic acid material is derived from a microorganism present in a microbiome from the subject.

43. The method of claim 42, wherein the sample is a fecal sample, and the microorganism is present in the gut microbiome of the subject.

44. The method of any one of claims 36 to 43, wherein the sample collection device is a swab that comprises, consists, or consists essentially of, a rod terminating in a tip portion with a non-absorbent surface.

45. The method of claim 44, wherein the sample drying agent is located at least partially in the container.

46. The use of any one of claims 36 to 45, wherein the sample drying element comprises a composition selected from activated alumina, aerogel, benzophenone, bentonite clay, calcium chloride, calcium oxide, calcium sulfate, cobalt(II) chloride, copper(II) sulfate, lithium chloride, lithium bromide, magnesium sulfate, magnesium perchlorate, potassium carbonate, potassium hydroxide, silica, sodium, sodium chlorate, sodium chloride, sodium hydroxide, sodium sulfate, sucrose, and sulfuric acid.

47. The use of claim 46, wherein the sample drying agent is housed in a sachet or packet.

48. The method of any one of claims 36 to 47, wherein the collection portion comprises a plurality of elongated fibres.

49. The method of claim 48, wherein the fibres have hydrophilic properties.

50. The method of claim 48 or claim 49, wherein the plurality of elongated fibres is arranged as a layer having a substantially uniform thickness.

51. The use of any one of claims 48 to 50, wherein the fibres are deposited on the tip portion by flocking on an ordered arrangement of the fibres normal to the non-absorbent surface.

52. The use of any one of claims 36 to 51, wherein the sample collection device is configured for collecting a sample selected from a fecal sample, a saliva sample, a skin sample, an oral sample, a genital sample, a nasal sample, an eye sample, and an ear sample.

53. The use of any one of claims 36 to 52, wherein the sample collected using the device is a fecal sample and used to characterise a microbiome (e.g., a gut microbiome).

54. A microbiome characterisation kit, the kit comprising a sample collection device, wherein the device comprises:
(a) a container;
(b) a collection element comprising a support body and a collection portion; and
(c) a sample drying agent the sample container, wherein the sample container is free from any sample processing reagents or chemicals, and configured to receive a sample from a collection site of the subject.

55. The kit of claim 54, wherein the kit further comprises instructions for collecting a sample that comprises microorganisms from a microbiome present in the subject.

56. The kit of claim 54 or claim 55, wherein the kit further comprises a return envelope or parcel for return of the sample to the nucleic acid sequencing facility.

57. A method for analysing a microbiome of a subject, comprising:
a. providing a sampling kit to a subject at a remote location, wherein the sampling kit comprises a sample collection device comprising (i) a container configured to receive a sample from a collection site of the subject; (ii) a collection element comprising a support body and a collection portion; and (iii) a sample drying agent;
b. receiving the sample container with the sample from the collection site of the subject;
c. generating a microbiome sequence dataset based upon sequencing nucleic acid content of at least one microorganism present in the sample;
d. identifying a set of microorganisms represented in the microorganism portion based upon performance of a mapping operation on portions of microbiome sequence dataset;
e. generating an analysis based upon a set of features related to the microorganism portion; and
f. transmitting information derived from the analysis to the subject.
